(19) United States
(12) Patent Application Publication
(10) Pub. No.: US 2026/0166059 A1
(43) Pub. Date: Jun. 18, 2026

(54) ORAL POUCH FOR ADMINISTRATION OF A CANNABINOID

(71) Applicant: Sensi Brands Inc., Richmond Hill (CA)

(72) Inventor: Antonio Giorgi, Stouffville (CA)

(73) Assignee: Sensi Brands Inc., Richmond Hill (CA)

(21) Appl. No.: 19/421,594

(22) Filed: Dec. 16, 2025

Related U.S. Application Data
(60) Provisional application No. 63/734,505, filed on Dec. 16, 2024.

(51) Int. Cl. A61K 31/00; A24B 13/00; A24B 15/16; A61K 9/00; A61K 9/16

(52) U.S. Cl. CPC A61K 31/658; A24B 13/00; A24B 15/16; A61K 9/009; A61K 9/1611; A61K 9/1617; A61K 9/1652; A61K 9/1694

(57) ABSTRACT

The present invention provides an oral cannabinoid pouch and process for preparing the oral cannabinoid pouch.

### FIELD OF THE INVENTION

The present invention pertains to the field of cannabinoid products and in particular to THC products.

### BACKGROUND

Prior art oral pouches for delivering tetrahydrocannabinol (THC) are made by coating commercially available flavor pouches with a THC distillate. This process results in an oral THC delivery product that does not provide consistent or long-lasting delivery of THC to the user.

This background information is provided to reveal information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide an oral pouch for administration of a cannabinoid. In accordance with an aspect of the present invention, there is provided a process for preparing an oral cannabinoid pouch, the process comprising: providing an oral pouch; providing a powdered cannabinoid filler formulation; introducing the powdered cannabinoid filler formulation into the oral pouch and sealing the pouch; providing a liquid cannabinoid infusion formulation; and applying the liquid cannabinoid infusion formulation to the filled oral pouch.

In accordance with another aspect of the present invention, there is provided an oral cannabinoid pouch for administration of cannabinoid comprising: an oral pouch; a powdered cannabinoid filler formulation inside the oral pouch; and a cannabinoid infusion formulation applied to the outside of the oral pouch.

### DETAILED DESCRIPTION OF THE INVENTION

The term “cannabinoid” is used to refer to the active constituents of cannabis, including tetrahydrocannabinol and cannabidiol.

The term “THC” is used to refer to the cannabinoid delta-9-tetrahydrocannabinol, or tetrahydrocannabinol, which is the principal psychoactive constituent of cannabis. THC can induce positive effects on mood of a person upon administration, including but not limited to feelings of euphoria or relaxation.

The term “CBD” is used to refer to the cannabinoid cannabidiol, which is a non-psychoactive constituent of cannabis.

As used herein, the term “about” refers to a +/−10% variation from the nominal value. It is to be understood that such a variation is always included in a given value provided herein, whether or not it is specifically referred to.

The term “oral pouch” is used to refer to a permeable bag or sachet, which is placed between the gum and the cheek or upper lip of the user.

The term “oral cannabinoid pouch” is used to refer to an oral pouch, containing cannabinoid formulations which, when the oral pouch is placed between the gum and the cheek or upper lip of the user, releases a cannabinoid into the bloodstream of the user.

The term “cannabis distillate” refers to a highly concentrated liquid extract created through the process of wiped film or molecular thin film distillation. Cannabis distillates contain high levels of cannabinoids, and a THC distillate can contain up to 99% pure THC.

Unless defined otherwise, all technical and scientific terms used herein have the same meaning as commonly understood by one of ordinary skill in the art to which this invention belongs.

The present invention provides an oral cannabinoid pouch which is suitable for administration of a cannabinoid via absorption through the oral mucosa into the bloodstream of a person. In accordance with an embodiment of the present invention, the oral cannabinoid pouch contains a cannabinoid powder inside the pouch and is also coated with a formulation comprising a cannabinoid. In embodiments, the cannabinoid powder contains THC, CBD, or a combination of THC and CBD.

The present invention also provides a two-stage process for preparing the oral cannabinoid pouch.

In one embodiment, the first stage of the process comprises filling an oral pouch with a powdered cannabinoid filler formulation, and the second stage of the process comprises applying a liquid cannabinoid infusion formulation to the filled oral pouch.

In one embodiment, the powdered cannabinoid filler formulation is prepared by combining a powdered cannabinoid composition with one or more excipients and a flavoring agent to form the powdered cannabinoid filler formulation. The resulting cannabinoid filler formulation is then introduced into an oral pouch. In one embodiment, to increase homogeneity of the resulting powdered cannabinoid filler formulation, the powdered cannabinoid filler formulation is further processed in a fluidized bed granulator prior to introduction into the oral pouch. This step facilitates formation of an active cannabinoid powder having an even particle size and even distribution of ingredients, and may be carried out using a fluidized bed granulator from Glatt.

In one embodiment, the powdered cannabinoid composition is formed by spray drying a first liquid cannabinoid composition. In one embodiment, conversion of the first liquid cannabinoid composition into the powdered cannabinoid filler formulation is carried out by feeding the liquid cannabinoid composition through spray drying equipment, where the liquid composition is converted into a dry powder through rapid drying with hot gas. The spray drying step can be carried out using a spray dryer, such as an EnviroStar rotary atomizing spray drying system.

In one embodiment, the first liquid cannabinoid composition is a liquid emulsion comprising a cannabinoid, water, an oil component and optionally a starch component. In one embodiment, the oil component of the liquid cannabinoid composition comprises a medium chain triglyceride (MCT) oil and/or a neutral oil, including but not limited to soybean oil, canola oil, grapeseed oil, sunflower oil and the like, and the starch component is a modified starch, such as acidulated corn starch.

In one embodiment, the cannabinoid in the composition is provided as a cannabis extract containing THC. In another embodiment, the cannabinoid in the composition is provided as a THC distillate or THC isolate.

In one embodiment, to form the liquid emulsion, the components of the first liquid cannabinoid composition are combined and converted into a liquid emulsion via high-speed shear mixing followed by homogenization. In one embodiment, the components of the emulsion comprise a first component and a second component in a ratio of about 1:1 to 1:10 of the first component to the second component. In one embodiment, the first component comprises from about 3% w/w to about 50% w/w of a concentrated cannabis extract/distillate, from about 0.5% w/w to about 15% w/w of the oil component, and from about 40% w/w to about 97% w/w of the starch component; and the second component comprises distilled water. The modified starch is provided to act as a carrier to encapsulate the distillate, turning liquid into powder. The oil component provides stability to active ingredients and solubilizes the cannabinoid extract/distillate.

The homogenization step can be carried out using a homogenizer, such as a Rannie homogenizer.

In an alternative embodiment, the first liquid cannabinoid composition is a solution comprising a cannabinoid and a solvent.

The powdered cannabinoid filler formulation may also contain excipients, including but not limited to artificial and/or natural flavours, pH stabilizers, and fillers such as microcrystalline cellulose.

The second stage of the process comprises preparing a liquid cannabinoid infusion formulation. The liquid cannabinoid infusion formulation is then applied to the oral pouch.

In one embodiment, the liquid cannabinoid infusion formulation is an oil-based liquid infusion. In one embodiment, the liquid cannabinoid infusion formulation comprises a mixture of cannabinoid, a flavoring agent and an oil component. In one embodiment, the liquid cannabinoid infusion formulation comprises THC, an oil-based flavoring agent and an oil component. In one embodiment, the oil component of the infusion formulation comprises a medium chain triglyceride (MCT) oil and/or a neutral oil.

In one embodiment, the cannabinoid in the infusion formulation is provided as a cannabis extract containing THC. In another embodiment, the cannabinoid in the infusion formulation is provided as a THC distillate.

In one embodiment, the liquid cannabinoid infusion formulation further comprises additional ingredients, including but not limited to, stabilizers and flavor components.

In one embodiment, the liquid cannabinoid infusion formulation is a flavored liquid THC infusion formulation.

In one embodiment, to form the liquid cannabinoid infusion formulation, the components of the formulation are combined and mixed until homogenous. The homogenization step can be carried out using a high-speed overhead mixer.

In order to ensure precise and consistent cannabinoid dosage, the liquid cannabinoid infusion formulation is applied to each oral pouch individually. This step ensures that, compared to the spraying and tumbling processes of the prior art, an even distribution and accurate dosage of the liquid TCH infusion is achieved.

In one embodiment, the process of preparing the oral cannabinoid pouch is carried out using automated equipment. In one embodiment, the automated equipment carries out the process steps in a sequential manner. For example, the pouch is first filled with the powdered cannabinoid filler formulation followed by application of the liquid cannabinoid infusion formulation. Automated production may include an automated dosing technology which applies a precise filler dose and infusion dose to each pouch.

In one embodiment, the oral cannabinoid pouch comprises from about 5 mg to about 10 mg of THC in the powdered cannabinoid filler formulation inside the pouch. In one embodiment, the infusion formulation on the oral cannabinoid pouch comprises from about 2 mg to about 5 mg of THC and other flavor ingredients.

The process of the present invention produces a flavorful and fast acting oral THC pouch that provides instant flavor release and THC effects within minutes.

The oral THC pouch of the present invention also exhibits a two-stage release of THC to the user, as the THC liquid infusion is first absorbed by the user, followed by release of the internal THC ingredients after saliva has moistened and dissolved the THC ingredients located in the interior of the pouch.

In one embodiment, the pouch material is a non-woven cellulose material. In one embodiment, the pouch is sealed under application of heat and pressure after filling with the powdered cannabinoid filler formulation.

The invention will now be described with reference to specific examples. It will be understood that the following examples are intended to describe embodiments of the invention and are not intended to limit the invention in any way.

### Preparation of the Powdered Cannabinoid Composition

### Formation of Emulsion

To prepare a cannabinoid-containing emulsion for the spray dry method of preparing the powdered cannabinoid composition, a cannabis distillate, modified starch, and MCT oil are combined and heated to a temperature of between about 50-75° C. If the starch is provided in powder format, mix it in warm water to prepare a 70% solution prior to adding to the cannabis and MCT oil mixture. If necessary, warm the cannabinoid distillate to between about 45-65° C. to liquefy before mixing with other ingredients.

Table 1 provides exemplary amounts for low, standard, and high THC emulsion formulations, including the dilution factor.

TABLE 1

| Liquid Emulsion Recipe | Low | Standard | High |
|---|---|---|---|
| Concentrated Cannabis extract (THC | 5% | 22% | 45% |
| Distillate/isolate) | | | |
| Oil Based Diluent (MCT or neutral oil) | 1% | 6% | 13% |
| Modified Starch (acidulated corn starch) | 96% | 72% | 42% |
| Dilution Factor (distilled water) | 1X | 4X | 10X |

Combine the mixture with a high-speed electric mixer for at least 30 minutes, then load the mixed material into the homogenizer and homogenize until the emulsion has formed, indicated by the formation of a milky liquid. The emulsion is ready for conversion to the filler powder by spray-drying.

### Spray Drying Method for Preparation of the Powdered Cannabinoid Composition

The homogenized emulsion is passed through a spray dryer, which converts the liquid into dried powder through heating to evaporate the water. The dried powder travels through a cyclone assembly for collection.

### Alternate Method for Preparation of the Powdered Cannabinoid Composition

The powdered cannabinoid composition can be created by directly combining cannabinoid distillate with a nano encapsulated acidulated cornstarch in a high-speed shear mixer for 5 mins.

### Preparation of Flavored Powdered Cannabinoid Filler Formulation

The powdered cannabinoid composition is tested to validate the content of THC or CBD prior to formulation of the powdered cannabinoid filler formulation to ensure the proper cannabinoid levels of the final formulation. The final formulation will be adjusted as required to ensure that a 0.45 g dose of the powdered cannabinoid filler formulation can provide 3 to 9 mg of THC per oral pouch.

The powdered cannabinoid composition is combined with microcrystalline cellulose, a flavor powder, sodium bicarbonate and magnesium stearate in the hopper of an agglomerator/mixer. Once the formulated material has been mixed for a 30-minute cycle, stop the equipment. The final formulated material is visually inspected to ensure no clumping is present, and that the material is uniformly mixed.

Table 2 provides exemplary amounts for low, standard, and high flavored powdered THC filler formulations.

TABLE 2

| Flavored powdered THC filler | | | |
|---|---|---|---|
| formulations (5 mg) | Low | Standard | High |
| Powdered THC composition | 25% | 4% | 1.00% |
| Natural Flavour | 0-75% | 20% | 0-75% |
| pH stabilizer (Sodium Bicarbonate) | 0-20% | 5% | 0-20% |
| Magnesium Stearate | 0-5% | 1% | 0-5% |
| Micro Crystlline Cellulose | 0-75% | 70% | 0-99% |

### Preparation of the Oral Pouch

The flavored powdered cannabinoid filler formulation is loaded into a hopper for automatic filling of a fabric pouch by a sachet filling machine. After filling, the pouch is sealed by the machine under heating and pressure. The filled pouches are stored for a minimum of 12 hours at a temperature of about 15-30° C. to cure prior to the next step of infusion.

### Preparation of Liquid Cannabinoid Infusion Formulation

A combination of cannabinoid distillate, MCT oil, and flavoring components is prepared and mixed until homogenized. The formulation of the infusion mixture is adjusted to ensure that a 0.2 g dose of the infusion formulation brings the potency of the total unit to 10 mg of THC.

Table 3 provides exemplary amounts for low, standard, and high flavored liquid THC infusion formulations.

TABLE 3

| Liquid THC infusion formulations | Low | Standard | High |
|---|---|---|---|
| Concentrated THC extract (THC | 0.50% | 2.50% | 5% |
| Distillate) | | | |
| Oil Based Diluent (MCT or neutral oil) | 0-99.5% | 67.5% | 0-95% |
| Oil Based Natural Flavour (natural | 0-75% | 20% | 0-75% |
| flavor agent in MCT or neutral oil | | | |
| Natural Oil Extract (Mint, Cinnamon, | 0-35% | 10% | 0-35% |
| Citrus, etc,) | | | |

### Preparation of the Coated Oral Pouch

The filled pouches are dosed with a total of 0.2 g of the formulated liquid. The dosed/infused are placed on a baking tray lined with paper for curing (˜1000 units per tray) for a minimum of 12 hours at a temperature of about 20-30° C., and a rH % between about 30-75%. This allows sufficient time for the liquid infusion to fully distribute throughout the individual oral pouch.

It is obvious that the foregoing embodiments of the invention are examples and can be varied in many ways. Such present or future variations are not to be regarded as a departure from the spirit and scope of the invention, and all such modifications as would be obvious to one skilled in the art are intended to be included within the scope of the following claims.

We claim:

1. A process for preparing an oral cannabinoid pouch, the process comprising:
providing an oral pouch;
providing a powdered cannabinoid filler formulation;
introducing the powdered cannabinoid filler formulation into the oral pouch and sealing the pouch;
providing a liquid cannabinoid infusion formulation; and
applying the liquid cannabinoid infusion formulation to the filled oral pouch.

2. The process of claim 1, wherein the powdered cannabinoid filler formulation is a flavored powdered cannabinoid filler formulation.

3. The process of claim 2, wherein the powdered cannabinoid filler formulation is prepared by combining a powdered cannabinoid composition with one or more excipients and a flavoring agent to form the powdered cannabinoid filler formulation.

4. The process of claim 3, wherein the one or more excipients include a filler, a pH stabilizer, and/or a stearate.

5. The process of claim 3, wherein the filler is microcrystalline cellulose.

6. The process of claim 3, wherein the pH stabilizer is sodium bicarbonate and the stearate is magnesium stearate.

7. The process of claim 3, wherein the powdered cannabinoid composition is prepared by spray drying a first liquid cannabinoid composition to form the powdered cannabinoid composition.

8. The process of claim 7, wherein the first liquid cannabinoid composition is a liquid emulsion comprising a cannabinoid, water, an oil component and a starch component.

9. The process of claim 8, wherein the liquid emulsion is formed via high-speed shear mixing, optionally followed by homogenization, of a first component and a second component, in a ratio of about 1:1 to 1:10 of the first component to the second component, wherein:
the first component comprises:
from about 3% w/w to about 50% w/w of a concentrated cannabis extract,
from about 0.5% w/w to about 15% w/w of the oil component, and
from about 40% w/w to about 97% w/w of the starch component; and
the second component comprises distilled water.

10. The process of claim 8, wherein the oil component of the liquid cannabinoid composition comprises a medium chain triglyceride (MCT) oil and/or a neutral oil.

11. The process of claim 7, wherein the first liquid cannabinoid composition is a solution comprising a cannabinoid and a solvent.

12. The process of claim 3, wherein the powdered cannabinoid composition is prepared by combining a cannabinoid distillate with a nano-encapsulated acidulated cornstarch by high shear mixing.

13. The process of claim 1, further comprising the step of processing the powdered cannabinoid filler formulation in a fluidized bed granulator prior to introduction into the oral pouch.

14. The process of claim 1, wherein the liquid cannabinoid infusion formulation comprises a cannabinoid and an oil component.

15. The process of claim 14, wherein the oil component of the liquid cannabinoid infusion formulation comprises a medium chain triglyceride (MCT) oil.

16. The process of claim 1, wherein the liquid cannabinoid infusion formulation further comprises one or more flavor components to provide a flavored liquid cannabinoid infusion formulation.

17. The process of claim 1, wherein the cannabinoid in the powdered cannabinoid filler formulation is THC.

18. The process of claim 1, wherein the cannabinoid in the cannabinoid infusion formulation is THC.

17. An oral cannabinoid pouch prepared using the process as defined in claim 1.

18. An oral cannabinoid pouch for administration of a cannabinoid comprising:
an oral pouch;
a powdered cannabinoid filler formulation inside the oral pouch; and
a cannabinoid infusion formulation applied to the oral pouch.

19. The oral cannabinoid pouch of claim 18, wherein the cannabinoid in the powdered cannabinoid filler formulation is THC.

20. The oral cannabinoid pouch of claim 18, wherein the cannabinoid in the cannabinoid infusion formulation is THC.
